(19) United States
(12) Patent Application Publication
(10) Pub. No.: US 2025/0186739 A1
(43) Pub. Date: Jun. 12, 2025

(54) METHODS AND SYSTEMS FOR USE WITH A CARDIAC PROCEDURE TO REDUCE POST OPERATIVE ATRIAL FIBRILLATION

(71) Applicant: David J. Ternes, Roseville, MN (US)

(72) Inventor: David J. Ternes, Roseville, MN (US)

(21) Appl. No.: 18/535,079

(22) Filed: Dec. 11, 2023

(51) Int. Cl. A61M 25/00; A61M 5/145; A61M 25/01

(52) U.S. Cl. CPC A61M 25/0084; A61M 5/14526; A61M 2025/0089; A61M 25/0108; A61M 2210/125

(57) ABSTRACT

One example provided a method for use with a cardiac procedure, including inserting a steerable delivery system into the body of a patient, the delivery system defining a lumen having an injection device disposed therein, and steering a distal end of the delivery system successively to each injection site of a plurality of cardiac injection sites, each injection site having a corresponding ganglionated plexi (GP) location. At each injection site, the method includes inserting a needle of the injection device into the injection site by extending the needle from a retracted position within the lumen to an extended position beyond the distal end, injecting a dosage volume of liquid treatment agent from the injection device into the injection site via the needle to treat the corresponding GP location to reduce potential occurrence of atrial fibrillation in response to the cardiac procedure, and retracting the needle into the lumen.

### BACKGROUND

Cardiac surgeries, such as transcatheter aortic valve replacement (TAVR) and coronary artery bypass grafts (CABG), for example, continue to become increasingly common. Atrial fibrillation (AF) is a common complication of cardiac surgery, affecting 20% to 50% of patients, and can lead to less favorable post-surgical outcomes, including prolonged hospitalizations and increased morbidity and mortality. Current approaches for treating post-surgical AF may introduce patient trauma in addition to that of the cardiac surgery.

For these and other reasons, a need exists for the present invention.

### BRIEF DESCRIPTION OF THE DRAWINGS

FIG. 1 is a flow diagram generally illustrating a method for use with a cardiac procedure to treat post-operative atrial fibrillation, according to one example.

FIG. 2A is a schematic diagram generally illustrating a portion of a human body of patient, according to one example.

FIG. 2B is a schematic diagram generally illustrating portion of a human heart, according to one example.

FIGS. 3A and 3B schematically illustrate portions of a human body, according to one example.

FIGS. 4A and 4B schematically illustrate portions of a human heart, according to examples of the present disclosure.

FIG. 5 is a block and schematic diagram generally illustrating a system for use with a cardiac procedure to treat post-operative atrial fibrillation, according to examples of the present disclosure.

FIGS. 6-6B are block and schematic diagrams generally illustrating an insertable medical device for use with a cardiac procedure to treat post-operative atrial fibrillation, according to examples of the present disclosure.

### DETAILED DESCRIPTION

In the following detailed description, reference is made to the accompanying drawings which form a part hereof, and in which are shown, by way of illustration, specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

AF is the most common type of cardiac arrhythmia (i.e., an irregular heartbeat). AF is due to abnormal electrical activity within the atria of the heart, causing them to fibrillate, and is often characterized as tachyarrhythmia (meaning that the heart rate is fast). Such arrhythmia may be paroxysmal (lasting less than seven days) or persistent (lasting more than seven days). The irregular rhythm of AF causes blood flow through the heart to become turbulent, resulting in an increased chance of forming a thrombus (blood clot), which can dislodge and cause a stroke. AF is the leading cardiac cause of stroke.

There are a wide variety of pathophysiological mechanisms that play a role in the development of AF, but cardiac remodeling accounts for the majority. Cardiac remodeling, both structural and electrical, particularly of atria, results in structural and electrical changes that eventually become the cause of deranged rhythm in AF. Structural remodeling is caused by changes in myocytes and the extracellular matrix, with fibrous tissue deposition also playing a key role in some etiologies. Electrical remodeling is caused by tachycardia and shortening of the refractory period.

While the underlying pathophysiology is not fully understood, hypertension, structural, valvular, and ischemic heart disease most commonly illicit the paroxysmal and persistent forms of AF. Most cases of AF are non-genetic and relate to underlying cardiovascular disease. Typically, an initiating trigger excites an ectopic focus in the atria, most commonly around the area of the pulmonary veins, and allows for an unsynchronized firing of electrical impulses leading to fibrillation of the atria. These impulses are irregular, and pulse rates can vary tremendously, leading to a turbulent and abnormal flow of blood through the heart chamber, which decreases the heart's effectiveness in pumping blood while increasing the likelihood of thrombus formation within the atria, most commonly in the left atrial appendage.

Unfortunately, AF is also often induced unintentionally by cardiac surgeries to treat other cardiac conditions, such as TAVR, CABG, aortic aneurysm repair, septal myectomies, and percutaneous coronary intervention (PCI), to name a few. Such postoperative AF (POAF) affects from 20% to 50% of surgical patients, and can negatively impact post-operative outcomes, including increased hospitalization times and increased mortality and morbidity. Although POAF has traditionally been considered self-limiting and relatively benign, up to 76% of these patients develop AF recurrences during the first postoperative year. POAF is associated with worse short- and long-term thromboembolic and survival outcomes, raising the need for long-term anticoagulation, especially after left-sided heart valve surgery.

In efforts to prevent POAF, several pharmacologic treatment agents, such as beta-blockers, sotalol, amiodarone, and anti-inflammatory agents, for example, have been tested and found to have varying efficacy, with their effect on length of stay (LOS), hospitalization costs, and long-term outcomes being mixed and remaining under debate. As a result, such treatments are weakly or moderately recommended as prophylactic therapies according to European guidelines.

Additional efforts to prevent POAF, at least in part, are currently being explored by a growing spectrum of autonomic neuromodulation therapies (ANMTs), including ganglionated plexus ablation, epicardial injections for temporary neurotoxicity, low-level vagus nerve stimulation (VNS), stellate ganglion block, baroreceptor stimulation, spinal cord stimulation, and renal denervation, for example. Such therapies are based on the anatomy and physiology of the autonomic nervous system (ANS) and its role in cardiac arrhythmias.

The ANS plays an important role in the initiation and development of AF, causing alterations in atrial structure and electrophysiological defects. Components of the peripheral, central, and intrinsic cardiac innervation systems form a complex interconnected network that manages cardiovascular function. The cardiac ANS is organized into extrinsic and intrinsic components that are supplied by the autonomic nerves. The intrinsic ANS of the heart comprises clusters of neurons known as ganglionated plexi (GP), typically embedded in epicardial adipose tissue (EAT), which form chain-like extensions that interconnect with the atria and ventricles as well as with the extrinsic cardiac ANS. These GPs contain both parasympathetic and sympathetic afferent and efferent neurological circuits that control the electrophysiological properties of the myocardium.

The intrinsic sympathetic innervation arises in the grey matter of the thoracis spinal cord segments T1-T6 and are generally myelinated fibers, that increase heart rate and myocardial contractility by releasing noradrenaline, stimulating inotropy in the heart. Parasympathetic fibers arise in the medulla oblongata, pons, and midbrain of the brainstem with some fibers arising from the sacral portion of the spinal cord (S2-S4). The resting heart is dominated by parasympathetic tone, which acts to reduce heart rate and slow cardiac impulses from the atria to the ventricles.

POAF has a multifactorial pathogenesis, central to which is autonomic dysfunction, systemic, peri- and myocardial inflammation, and oxidative stress in patients with an altered atrial substrate due to atrial ischemia, surgical injury, and comorbidities. Specifically, elevated postoperative blood noradrenaline levels, an increase in sinus rate, and hyperactivity of the major atrial GP preceding the initiation of POAF, point to a role for increased sympathetic tone. Sympathetic predominance is accompanied by increased vagal activity, indicating a combined sympatho-vagal triggering of POAF. ANMT is being investigated for POAF prevention due to its potential to suppress the autonomic overdrive and down-regulate inflammation.

During cardiac surgery and the postoperative period, the effect of acute arrhythmogenic factors related to cardiac surgery is superimposed on pre-existing AF risk factors (e.g., heart failure, hypertension, aging, diabetes, lung disease). POAF develops when the combined effect of the acute factors and the chronic AF risk factors exceeds a certain AF threshold. The intent of ANMTs is to prevent or reduce occurrence of POAF by attenuating the effect of these transient arrhythmogenic factors combined with the pre-existing AF risk factors.

GP activity affecting the pulmonary veins is another important factor in the pathogenesis of AF, with AF sources being found to co-localize with a GP location in many patients with paroxysmal AF. Pulmonary vein isolation (PVI), and other types of cardiac ablations, including GP ablation, can directly damage GPs, or disrupt the fibers connecting GPs, and thereby interrupt or block irregular electrical impulses to restore a regular heartbeat.

GP ablation has been evaluated as an adjunctive procedure for the treatment of patients suffering from AF. While GP ablation adjuvant to PVI has been found to improve outcomes in patients with paroxysmal AF, other studies have found that epicardial PVI alone provides no benefit in POAF prevention. Such outcomes imply that autonomic triggers play a critical role in POAF pathogenesis, while pulmonary vein firing plays a minor role, and suggest that an ablative strategy focusing on GP rather than pulmonary veins could target the upstream triggers of POAF and provide a more tailored approach for POAF prevention. However, performing an invasive adjunctive GP ablation procedure separately from the primary cardiac surgery subjects a patient to additional trauma (e.g., extended surgical durations, additional bodily incisions, cardiac tissue ablation) and may unintentionally increase the probability of POAF over that of the primary cardiac surgery (e.g., TAVR and CABG).

As will be described in greater detail herein, the present application discloses methods and systems to therapeutically treat atrial tissue to reduce the occurrence of POAF which may otherwise be unintentionally induced by cardiac surgery. In examples, the methods and systems described herein are suitable for minimally invasive surgery. In particular, the systems and methods described herein include treating GP sites located within cardiac tissue with injections of a treatment agent to induce neurotoxicity to inhibit hyperactivity of the cardiac ANS and reduce occurrence of POAF. In examples the treatment agent includes calcium chloride (CaCl₂) or botulinum toxin, for example.

In accordance with the present disclosure, such injections can be readily incorporated into surgical workflows of cardiac procedures and be performed temporally therewith, such as with TAVR and CABG procedures, for example, thereby eliminating the need for an entirely independent procedure and reducing surgical time and patient trauma. Such injections also reduce trauma to GP tissue relative to GP ablation procedures. The method, systems, and devices disclosed herein provide injections focused on treating GP sites and target upstream triggers of POAF to provide a more tailored and less traumatic approach for POAF prevention.

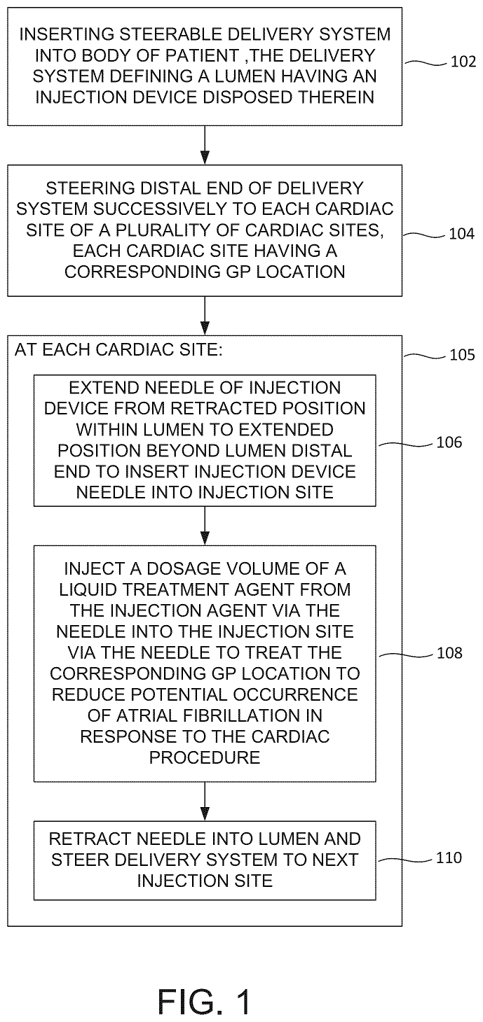

FIG. 1 is a flow diagram generally illustrating a method 100, according to the examples of the present disclosure, for use with a cardiac surgical procedure to reduce the potential occurrence of POAF being induced thereby. At 102, method 100 includes inserting a steerable delivery system into the body of a patient undergoing the cardiac surgical procedure (sometimes referred to herein as the principal cardiac surgery). In examples, as described below, the steerable delivery system is inserted into the cardiovascular system of the patient. In other examples, the delivery system is inserted into the thoracic cavity of the patient.

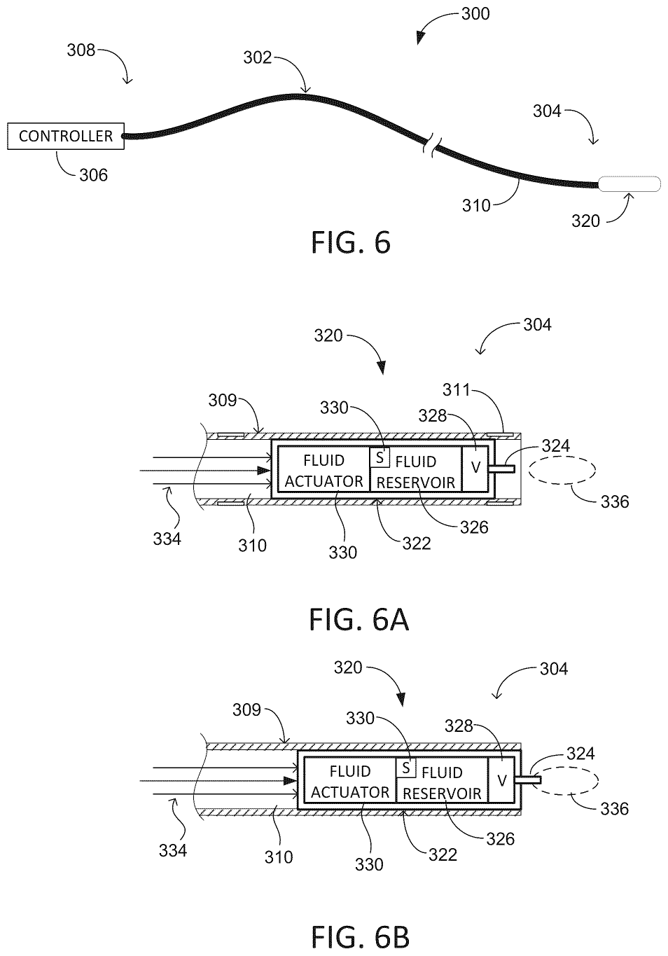

In examples, the steerable delivery system defines a lumen having an injection device disposed therein. In examples, the steerable delivery system comprises a catheter having a sheath defining the lumen, and having a steerable distal end, wherein the injection device includes a needle which is extendable and retractable from the lumen at the distal end (e.g., see FIGS. 6-6B below).

In examples, as mentioned above, the method (as well as the system and device), as disclosed herein, can be incorporated within or as part of a surgical workflow of a principal cardiac surgery. For example, the method can be integrated into the surgical workflow of a TAVR surgery, where the steerable delivery system of the present method is inserted into the patient's cardiovascular system via a same entrance incision made to as part of the workflow of the TAVR surgery, such as an incision into the patient's femoral artery, for instance.

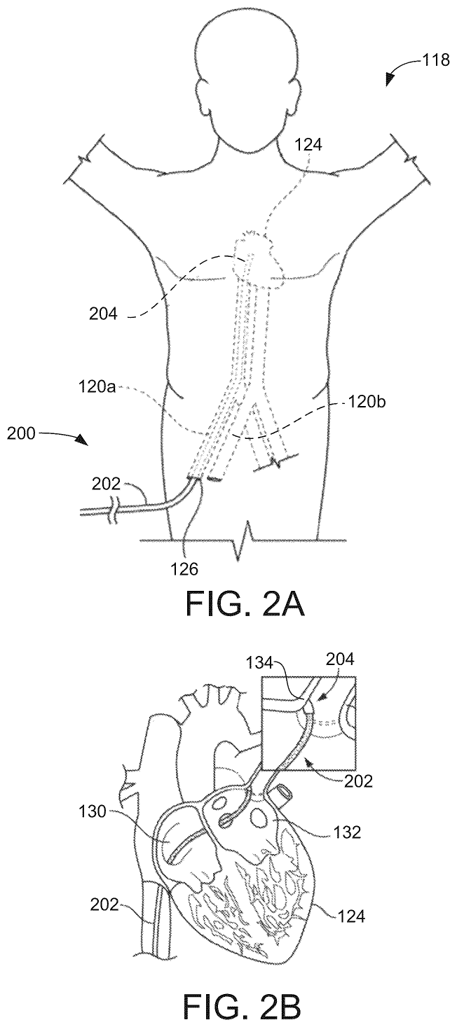

With reference to FIG. 2A, portions of a human body 118 (i.e., a patient) are diagrammatically illustrated, including right and left femoral arteries 120a and 120b, aorta 122, and heart 124. As illustrated, an insertable therapeutic medical device 300 including a steerable delivery system 302, according to examples of the present application (e.g., see FIGS. 6-6B below), is inserted into the cardiovascular system of patient 118 via an incision 126 in femoral artery 120a, and advanced through femoral artery 120a and aorta 122 so that a distal end 304 (e.g., a steerable distal end) is positioned within heart 124. In examples, a sheath and expander system employed to provide access to the cardiovascular system (e.g., the femoral artery) for the principal cardiac surgery (e.g., a TAVR surgery) is also utilized to provide access for the method and therapeutic device of the present disclosure, thereby simplifying the process and reducing patient trauma.

FIG. 2B illustrates advancement of steerable distal end 304 of delivery system 302 of therapeutic medical device 300 through right atrium 130, across a septum, and into left atrium 132 of heart 124 to an injection site 134 (e.g., a GP location).

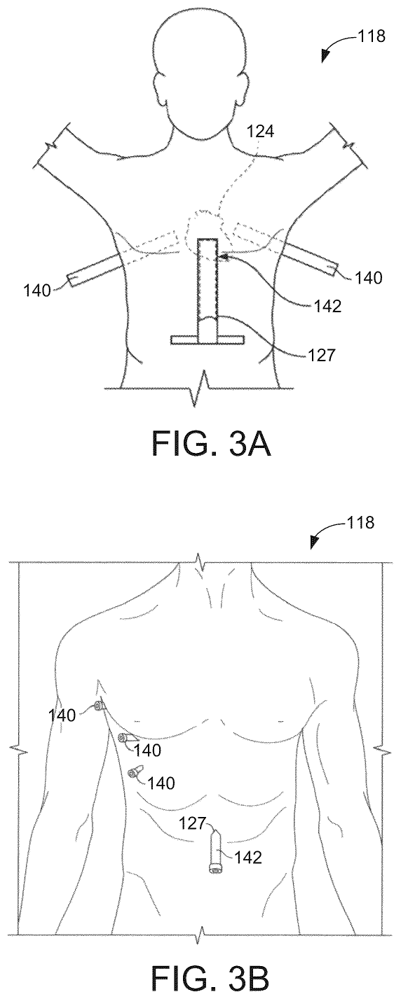

In some examples, with reference to FIGS. 3A and 3B, steerable delivery system 302 of therapeutic medical device 300, in accordance with the present disclosure, may be inserted into the thoracic cavity of patient 118 via one or more trocars 140 extending through the chest of patient 118 and/or via an access device 142 (e.g., a separator or elevator) inserted through an incision 127 in the abdomen of patient 118 as part of a principal cardiac procedure (e.g., a CABG procedure).

Returning to FIG. 1, according to examples, after insertion of steerable delivery system 302 into the cardiovascular system of patient 118, method 100, at 104, includes successively steering distal end 304 of delivery system 302 to each injection site of a plurality of cardiac injection sites of heart 124, where each injection site has a corresponding GP location. In examples, a physician steers distal end 304 (i.e., the working end of device 300) using any number of various modalities which enable a position of distal end 302 to determined (e.g., viewed) from the exterior of heart 124 and/or patient 118, such as fluoroscopy or ultrasound techniques, for instance.

In examples, prior to inserting delivery system 302 into body of patient 118, method 100 further includes a process for determining and marking the locations of each injection site of the plurality of cardiac injection sites. In one example, such injection site locating process includes determining location of the corresponding GP location of each injection and, from such GP location, determining the location of each injection site. For instance, in some examples, the injection site is a vein which is upstream of and provides blood flow to the corresponding GP site. In some examples, the injection site is coincident with the corresponding GP site, such that the injection site is the cardiac tissue of the corresponding GP site.

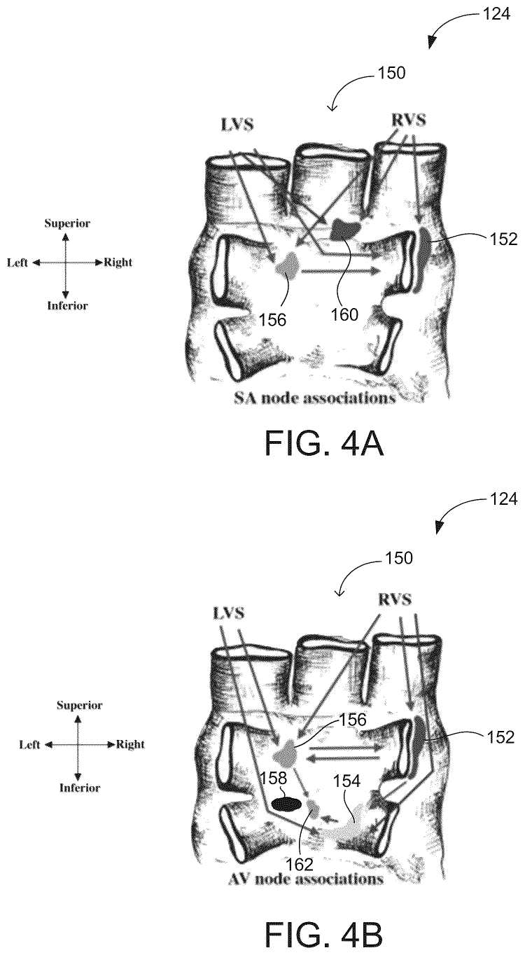

FIGS. 4A and 4B generally illustrate posterior views of the atria 150 of a heart, such as heart 124, and graphically depict locations of the anterior right GP (ARGP) 152, inferior right GP (IRGP) 154, superior left GP (SLGP) 156, inferior left GP (ARGP) 158, the sinoatrial (SA) node 160, and the atrioventricular (AV) node 162. The SA node generates electrical signals that cause the upper atria (heart chambers) to contract, with the signal passing through the AV node to the lower ventricles (heart chambers), causing them to contract, or pump. The AV node is disposed between the atria and ventricles and introduces a delay between atrial and ventricular excitation, allowing for efficient ventricular filling. Additionally, FIGS. 4A and 4B schematically illustrate interactions between neural pathways.

In FIG. 4A, arrows illustrate the direction of impulse and connections of the left vagosympathetic (LVS) nerve trunk and right vagosympathetic (RVS) nerve trunk with the ARGP 152 and SLGP 156. These pathways have been shown to modulate the effects of the SA node 160 and attenuate sinus rate slowing. In FIG. 4B, arrows illustrate the direction of impulse and connections involving the LVS and RVS nerve trunks with the SLGP 156, ARGP 152, and IRGP 154. These pathways have been shown to influence AV node 162 function and ventricular rate response. The ILGP 158 acts as a pivotal element in the connection pathway of the AV node. For additional reference, see also, for example, Ganglionated Plexi Ablation for the Treatment of Atrial Fibrillation, J. Clin. 3020 October; 9(10): 3081.

As described above, GPs are localized neural clusters of intrinsic cardiac ganglia, containing local circuits, parasympathetic neurons, and sympathetic afferents and efferents. The variety of neuronal contributions associated with each ganglion reflects their complex synaptology. GPs typically contain 200 to 1000 neurons and are variable in size, with predominantly oval-shaped soma. Histological studies show the mean area of a human ganglia to be 0.07±0.02 mm², with few exceeding 0.2 mm². GPs are typically found embedded in epicardial adipose tissue (EAT) and have been described as having a “raisin in bread” pattern, forming chainlike extensions onto the atria and ventricles. The degree of EAT coverage varies in quantity and depth and is generally concentrated along the coronary sulcus and interventricular and atrioventricular grooves. For additional reference, see also, for example, Ganglionated Plexi Ablation for the Treatment of Atrial Fibrillation, J. Clin. 3020 October; 9(10): 3081.

In one example, a technique which may be employed by the present method to determine GP locations, which uses endocardial high frequency stimulation (HFS), is described by Is Vagal Response During Left Atrial Ganglionated Plexi Stimulation a Normal Phenomenon?, Circ Arrhythm Electrophysiol. October 2019, 1-9. According to such technique, endocardial HFS stimulation (e.g., using a MicroPace EPS320 cardiac stimulator; MicoPace EP, Inc; providing a 20 Hz, 25 mA output having a 10 ms pulse duration) is applied at presumed anatomic areas of each of the GPs selected to be treated. At the presumed anatomic area of each GP, the position of the cardiac stimulator can be controllably moved about until a vagal response is observed, where such vagal response is defined as a prolongation of the R-R interval relative to a pre-HFS R-R interval, and is indicative of the location of the corresponding GP. The position of the GP can then be used to identify and locate a cardiac injection site for the present method 100. As described above, in some examples, the cardiac injection site may be a vein upstream of the GP location. In other examples, the cardiac injection site may be the GP itself.

In some examples, prior to the application of the endocardial HFS, the location technique further includes performance of a mapping procedure to construct a 3-demensional image of the heart, where such mapping, according to one example, includes use of the CARTO 3 mapping system by Biosense Webster, Diamond Bar, CA; and use of a multipole, multispline mapping catheter (e.g., 2-6-2-mm inter-electrode spacing, PentaRay NAV; Biosense Webster). Is Vagal Response During Left Atrial Ganglionated Plexi Stimulation a Normal Phenomenon?, Circ Arrhythm Electrophysiol. October 2019, 1-9.

Returning to FIG. 1, upon steering the distal end of the delivery system to a cardiac injection site (e.g., distal end 204 of delivery system 202), where such injection site location may be determined using the above-described HFS and 3-dimensional imaging techniques, at each injection site, method 100, at 106, includes inserting a needle of the injection device into the injection site. In one example, inserting the needle into the injection site is performed by extending the needle to a retracted position within the lumen to an extending position beyond the distal end of the lumen, such as from distal end 204 of delivery system 202.

Additionally, according to one at example, upon inserting the needle into the injection site, method 100, at 108, includes injecting a dosage volume of a liquid treatment agent from the injection device into the injection site to treat the corresponding GP location to reduce the potential occurrence of atrial fibrillation in response to the cardiac procedure being performed (i.e., to reduce the potential occurrence of POAF in response to the principal cardiac surgery). In examples, the liquid treatment agent may be one of botulinum toxin or calcium chloride (CaCl₂) which have been shown to induce neurotoxicity at GP locations, which inhibits hyperactivity of the cardiac ANS. In other examples, suitable liquid treatment agents which induce such neurotoxicity, other than botulinum toxin and CaCl₂, may be employed.

In one example, the dosage volume of the liquid treatment agent injected at each injection site is controllable such that the volume of injected treatment agent may vary between GP locations. In another example, each time the injection device is activated to eject the treatment agent, the injection device dispenses a same volume of liquid treatment agent via the needle. According to such example, the injection device may be activated a different number of times as each GP location in order to vary the total volume of treatment agent (i.e., the dosage volume) injected at different GP locations. In some examples, a dosage volume of liquid treatment agent to be injected at a given GP location may be determined based on associated electrical signals, such as the level of R-R interval alteration a given GP location may exhibit in response to endocardial HFS during the GP mapping and locating process. For example, the greater the R-R interval alteration, the higher the dosage volume.

With continued reference to FIG. 1, method 100 concludes at 110, where the needle of the injection device is returned to the retracted position within the lumen, such as within distal end 204 of delivery system 202. According to examples, the distal end of the delivery system is then steered to the next injection site, or is removed from the body of the patient if the injections have been completed at each of the injection sites.

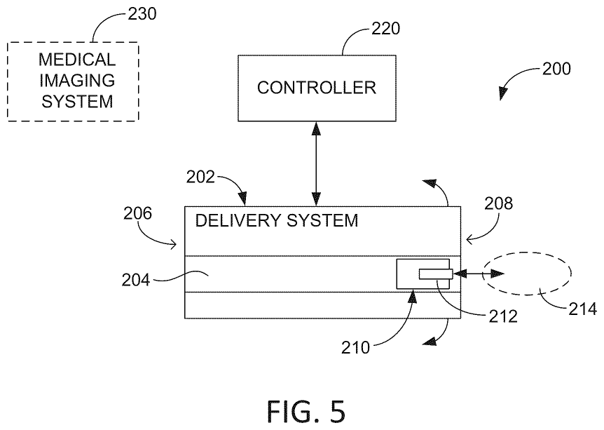

FIG. 5 is a block and schematic diagram generally illustrating a system 200 for use with a cardiac procedure to implement the above-described method of FIG. 1, according to one example of the present disclosure. According to one example, system 200 includes a delivery system 202 which is insertable with the body of a patient, such as into the patient's cardiovascular system. In examples, the delivery system defines a lumen 204 and has a proximal end 206 and a distal end 208, where distal end 208 is steerable (as indicated by arrows).

System 200 further includes an injection device 210 disposed within lumen 204, where injection device 210 includes a needle 212 which is moveable between a retracted position within lumen 204 and an extended position beyond distal end 208 of delivery system 202 (as indicated by the double ended directional arrow). In examples, upon distal end 208 being positioned at a selected injection site 214 of the patient's heart, the selected injection site 214 having a corresponding GP location, injection device 210 is controlled to move needle 212 to the extended position to be inserted into selected injection site 214 and inject a dosage of a liquid treatment agent therein to treat the corresponding GP location to reduce the potential occurrence of AF in response to the cardiac procedure (e.g., a TAVR procedure). In examples, the liquid treatment agent comprises one of botulinum toxin and CaCl₂.

In one example, system 200 further includes a controller 220 in mechanical and/or electrical communication with delivery system 202 and injection device 210, wherein controller 220 is configured to steer distal end 208 of delivery system 202 to selected injection site 214, to move needle 212 to the extended position to insert needle 212 into selected injection site 214, and to operate injection device 210 to inject the liquid treatment agent into the selected injection site via needle 212.

In one example, system 200 is configured for use with a medical imaging system 230 which provides continuous imaging of delivery system 202 and injection device 210 to assist in steering of distal end 208 to injection site 214 and the insertion of needle 212 therein. In examples, medical imaging system may comprise a fluoroscopy system or an ultrasound imaging system. However, system 200 may be adapted for use with any number of various medical imaging systems other than, or in addition to, fluoroscopy and ultrasound systems.

FIGS. 6-6B are block and schematic diagrams generally illustrating an insertable therapeutic medical device 300 for use in concert with a cardiac procedure and to carry out the above-described method 100 to inject a liquid treatment agent at selected injection sites of a patient's heart to treat selected GP locations to reduce potential occurrence of POAF in response to the cardiac procedure, according to examples of the present disclosure.

With reference to FIG. 6, medical device 300 includes a flexible delivery system 302 having a steerable distal end 304 and a controller 306 at a proximal end 308. In examples, flexible delivery system 302 defines a lumen 310 and includes an injection device 320 disposed within the lumen at distal end 304. In one example, flexible delivery system 302 comprises a steerable catheter.

FIGS. 6A and 6B are block and schematic diagrams illustrating an enlarged view of distal end 304 of delivery system 302, and respectively illustrate injection device 320 in retracted and extends positions. In examples, flexible delivery system 302 includes a sheath 309 which defines lumen 310, where injection device 320 is disposed within lumen 310 at distal end 304.

In examples, injection device 320 includes a housing 322, a needle 324 extending for a distal end of injection device 320, a fluid reservoir 326 for storing the liquid treatment agent, a fluid control mechanism 328 disposed between fluid reservoir 326 and needle 324, a fluid actuator 330, and a fluid level sensor 332. In examples, a plurality of mechanical and electrical control inputs/outputs 334 extend between controller 306 and injection device 320 and steerable distal end 304 of delivery system 302.

In examples, as illustrated by FIG. 3A, while steerable delivery system 302 is being moved within the patient, such as between injection sites, for example, injection device 300 is moved to and maintained at a retracted position such that needle 324 is disposed within the distal end of lumen 310. In examples, delivery system 202 includes a number of fiducial markers, as illustrated by fiducial marker 311, which enable delivery system 302 to be tracked by an external medical imaging system (e.g., see FIG. 5) to assist in steering of distal end 304 to a selected injection site 336.

In examples, as illustrated by FIG. 3B, when distal end 304 is positioned at a selected injection site 336, injection device 320 is controlled via controller 306 to move from the retracted position to an extended position to insert needle 324 within the selected injection site 336. In examples, as illustrated, housing 322 of injection device 320 is controllably moved back and forth within sheath 309 to extend and retract needle 324 from the distal end of lumen 310.

In examples, once needle 324 is inserted within selected injection site 336, fluid actuator 330 is operated via controller 306 to inject a dosage volume of liquid treatment agent (e.g., botulinum toxin, CaCl₂, or other suitable treatment agent) from fluid reservoir 326 through fluid control mechanism 328 and needle 324 into injection site 336. In some examples, injection site 336 may be a GP location. In some examples, injection site 336 may be a blood vessel disposed upstream of a corresponding GP location so that the liquid treatment agent is carried to the corresponding GP location via the blood stream.

In examples, fluid control mechanism 328 provides directional control of liquid treatment agent so that the treatment agent or bodily fluids are unable to flow into fluid reservoir 326 via needle 324. In one example, fluid control mechanism 326 comprises a one-way valve to prevent backflow into fluid reservoir 326.

In one example, fluid actuator 330 is controllable via controller 306 to inject selected dosage volumes of liquid treatment agent from reservoir 326. For example, in one example, fluid actuator 330 comprises an electric motor to operate a pump or a plunger to eject liquid treatment agent from reservoir 326 via fluid control mechanism 328 and needle 324, wherein an electric control signal from controller 306 is adjustable to control the dosage volume (e.g., a voltage level, current level, and/or duration of the electric control signal (including digital signals) controls the electric motor to dispense a selected dosage volume). In some examples, fluid actuator 330 may be operated via mechanical controls extending from controller 306 (e.g., control cables and air pressure). In examples, electrical control signals may be transmitted wirelessly (e.g., Bluetooth) to injection device 320.

In examples, a fluid sensor 332 monitors a fluid level within fluid reservoir 326, wherein the fluid level readings provided by fluid sensor 332 may be used to determine the dosage volume of liquid treatment agent dispensed by each operation of fluid actuator 330 and to monitor an amount of liquid treatment agent present stored within reservoir 326. In other examples, each time fluid actuator 330 is operated, fluid actuator 330 is configured to dispense a same dosage volume of liquid treatment agent from fluid reservoir 326, whereby multiple injections can be made at a same injection site 336 so that the dosage of liquid treatment agent injected at each injection site can be varied.

It is to be understood that the features of the various exemplary embodiments described herein may be combined with each other, unless specifically noted otherwise.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

What is claimed is:

1. A method for use with a cardiac procedure, comprising:
inserting a steerable delivery system into the body of a patient, the delivery system defining a lumen having an injection device disposed therein;
steering a distal end of the delivery system successively to each injection site of a plurality of cardiac injection sites, each injection site having a corresponding ganglionated plexi (GP) location;
at each injection site, the method including:
inserting a needle of the injection device into the injection site by extending the needle from a retracted position within the lumen to an extended position beyond the distal end; and
injecting a dosage volume of liquid treatment agent from the injection device into the injection site via the needle to treat the corresponding GP location to reduce potential occurrence of atrial fibrillation in response to the cardiac procedure; and
retracting the needle into the lumen.

2. The method of claim 1, wherein the injection site comprises a bloodstream upstream of the corresponding GP location.

3. The method of claim 1, wherein the liquid treatment agent comprises one of botulinum toxin and CaCl₂.

4. The method of claim 1, wherein the dosage volume is selectable.

5. The method of 1, including varying the dosage volume between injection sites.

6. The method of claim 1, where inserting the steerable delivery system into the body of the patient includes inserting the steerable delivery system into a cardiovascular system of the patient.

7. The method of claim 1, where inserting the steerable delivery system into the body of the patient includes inserting the steerable delivery system into a thoracic cavity and/or abdomen of the patient.

8. The method of claim 1, including incorporating the method into a surgical workflow of the cardiac procedure such that the method is performed before the cardiac procedure, during the cardiac procedure, or after the cardiac procedure.

9. The method of claim 1, wherein the cardiac procedure comprises one of a transcatheter aortic valve replacement (TAVR) procedure and a coronary artery bypass grafts (CABG) procedure.

10. The method of claim 1, including identifying locations of each injection site of the plurality of cardiac injection sites based on identifying and marking a location of the corresponding GP site.

11. The method of claim 1, wherein injecting the dosage volume of liquid treatment agent includes injecting the liquid treatment agent from a reservoir within the injection device holding a total volume of liquid treatment agent which is greater than the dosage volume.

12. The method of claim 11, including monitoring the volume of liquid treatment agent in the reservoir.

13. The method of claim 1, wherein steering the distal end of the delivery system and inserting the needle of the injection device into the first selected injection site includes using a medical imaging system to provide images to guide the delivery system and the needle within the cardiovascular system.

14. The method of 13, including providing fiducial markers on the delivery system and the injection device which are visible via the images provided by the medical imaging system.

15. A system for use with a cardiac procedure, comprising:
a delivery system insertable within a body of a patient, the delivery system defining a lumen and having a proximal end and a distal end, the distal end being steerable; and
an injection device disposed within the lumen, the injection device including a needle moveable between a retracted position within the lumen and an extended position beyond the distal end of the delivery system, upon the distal end of the delivery system being positioned at a selected injection site, the selected injection site having a corresponding ganglionated plexi (GP) location, the injection device controlled to move the needle to the extended position to be inserted into the selected injection site and to inject a dosage volume of a liquid treatment agent into the selected injection site to treat the corresponding GP location to reduce potential occurrence of atrial fibrillation in response to the cardiac procedure.

16. The system of claim 15, wherein the liquid treatment agent comprises one of botulinum toxin and CaCl₂.

17. The system of claim 15, the injection device including:
a reservoir to hold a liquid treatment agent; and
a fluid actuator operable to inject the dosage volume of liquid treatment agent from the reservoir through the needle.

18. The system of claim 17, wherein the reservoir holds a total volume of liquid treatment agent greater than the dosage volume.

19. The system of claim 15, the injection device including a controller to:
steer the distal end of the delivery system to a selected injection site having a corresponding ganglionated plexi (GP) location;
move the needle to the extended position to insert the needle into the selected injection site; and
operate the fluid actuator to inject a dosage volume into the selected injection site to reduce potential occurrence of atrial fibrillation in response to the cardiac procedure.

20. The system of claim 19, wherein the controller includes mechanical and/or electrical control connections between the delivery system and the injection device.

21. The system of claim 15, the delivery system and injection device each including fiducial markers visible in images provided by a medical imaging system to enable steering and positioning of the delivery system and needle via the medical imaging system.

22. An insertable therapeutic medical device for use in concert with a cardiac procedure, comprising:
a delivery system insertable within the human body, the delivery system defining a delivery lumen having a proximal end and a distal end, the distal end being steerable; and
an injection device disposed within the distal end of the delivery lumen, the device including:
a needle having a shaft and tip with a needle lumen extending there through, wherein at least the tip is moveable between a retracted position, where the tip is disposed within the delivery lumen, and an extended position where the tip is disposed beyond the distal end of the delivery lumen;
a reservoir to hold a volume of an injectable liquid treatment agent, the reservoir in fluid communication with the needle lumen; and
a fluid actuator controllable to inject a dosage volume of the liquid treatment agent through the needle lumen and into a selected injection site during an injection operation when the needle tip is inserted therein to treat a corresponding GP location to reduce potential occurrence of atrial fibrillation in response to a cardiac procedure.

23. The therapeutic medical device of claim 22, wherein the liquid treatment agent comprises one of botulinum toxin and CaCl₂.

24. The therapeutic medical device of claim 22, wherein the fluid actuator is controllable to adjust the dosage volume.
